(19) United States
(12) Patent Application Publication
(10) Pub. No.: US 2024/0078666 A1
(43) Pub. Date: Mar. 7, 2024

(54) SYSTEMS, METHODS, AND APPARATUSES FOR IMPLEMENTING PATCH ORDER PREDICTION AND APPEARANCE RECOVERY (POPAR) BASED IMAGE PROCESSING FOR SELF-SUPERVISED LEARNING MEDICAL IMAGE ANALYSIS

(71) Applicant: Arizona Board of Regents on behalf of Arizona State University, Scottsdale, AZ (US)

(72) Inventors: Jiaxuan PANG, Mesa, AZ (US); Fatemeh Haghighi, Tempe, AZ (US); DongAo Ma, Mesa, AZ (US); Nahid Ui Islam, Mesa, AZ (US); Mohammad Reza Hosseinzadeh Taher, Tempe, AZ (US); Jianming Liang, Scottsdale, AZ (US)

(21) Appl. No.: 18/241,809

(22) Filed: Sep. 1, 2023

Related U.S. Application Data
(60) Provisional application No. 63/403,609, filed on Sep. 2, 2022.

(51) Int. Cl. G06T 7/00; G06T 7/11; G06V 10/54; G16H 30/40

(52) U.S. Cl. CPC G06T 7/0012; G06T 7/11; G06V 10/54; G16H 30/40; G06T 2207/20081; G06V 2201/03

(57) ABSTRACT

A self-supervised machine learning method and system for learning visual representations in medical images. The system receives a plurality of medical images of similar anatomy, divides each of the plurality of medical images into its own sequence of non-overlapping patches, wherein a unique portion of each medical image appears in each patch in the sequence of non-overlapping patches. The system then randomizes the sequence of non-overlapping patches for each of the plurality of medical images, and randomly distorts the unique portion of each medical image that appears in each patch in the sequence of non-overlapping patches for each of the plurality of medical images. Thereafter, the system learns, via a vision transformer network, patch-wise high-level contextual features in the plurality of medical images, and simultaneously, learns, via the vision transformer network, fine-grained features embedded in the plurality of medical images.

### CROSS REFERENCE TO RELATED APPLICATIONS

The present application claims priority under 35 U.S.C. § 119(e) to U.S. Provisional Patent Application No. 63/403,609, filed Sep. 2, 2022, and U.S. Provisional Patent Application No. 63/403,596 filed Sep. 2, 2022, the entire contents of each of which are hereby incorporated by reference. This non-provisional application is related to U.S. Non-Provisional Patent Application Number ##/###, ### filed Sep. 1, 2023, entitled “SYSTEMS, METHODS, AND APPARATUSES FOR IMPLEMENTING DISCRIMINATIVE, RESTORATIVE, AND ADVERSARIAL (DiRA) LEARNING USING STEPWISE INCREMENTAL PRE-TRAINING FOR MEDICAL IMAGE ANALYSIS”, the entire contents of which are incorporated herein by reference.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate generally to the field of medical imaging and analysis using convolutional neural networks for the classification and annotation of medical images, and more particularly, to systems, methods, and apparatuses for implementing Patch Order Prediction and Appearance Recovery (POPAR) based image processing for self-supervised learning medical image analysis.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to embodiments of the claimed inventions.

Machine learning models have various applications to automatically process inputs and produce outputs considering situational factors and learned information to improve output quality. One area where machine learning models, and neural networks in particular, provide high utility is in the field of processing medical images.

Within the context of machine learning and with regard to deep learning specifically, a Convolutional Neural Network (CNN, or ConvNet) is a class of deep neural networks, very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

The present state of the art may therefore benefit from the systems, methods, and apparatuses for implementing Patch Order Prediction and Appearance Recovery (POPAR) based image processing for self-supervised learning medical image analysis, as described herein.

### BRIEF DESCRIPTION OF THE DRAWINGS

Embodiments are illustrated by way of example, and not by way of limitation, and can be more fully understood with reference to the following detailed description when considered in connection with the figures in which:

FIGS. 1A and 1B depict typical photographic images having objects of considerable differences on varying backgrounds compared with medical images having similar appearances;

FIGS. 2A, 2B, and 2C depict an exemplary POPAR architecture, in accordance with described embodiments;

FIG. 3 depicts Table 1 which shows an evaluation of POPAR with ViT-B and Swin-B backbones, in accordance with described embodiments;

FIG. 4 depicts Table 2 which shows that POPAR models experimentally outperform other known state-of-the-art self-supervised ImageNet models with transformer backbone in three target tasks, in accordance with described embodiments;

FIG. 5 depicts Table 3 which shows that POPAR models yield significant performance boosts over other known state-of-the-art techniques, in accordance with described embodiments;

FIG. 6 depicts Table 4 which shows that POPAR models were experimentally shown to outperform fully supervised pre-trained models on ImageNet and ChestX-ray14 datasets in three target tasks across architectures, in accordance with described embodiments;

FIG. 7 depicts Table 5 which shows that each component in POPAR is necessary, in accordance with described embodiments; and

FIG. 8 depicts equations as utilized in conjunction with the POPAR framework and trained POPAR models, in accordance with described embodiments.

### DETAILED DESCRIPTION

Described herein are systems, methods, and apparatuses for implementing Patch Order Prediction and Appearance Recovery (POPAR) based image processing for self-supervised learning medical image analysis.

In the field of medical image analysis, vision transformer-based self-supervised learning (SSL) approaches have recently shown substantial success in learning visual representations from unannotated photographic images. However, their acceptance in medical imaging is still lukewarm, due to the significant discrepancy between medical and photographic images. Consequently, embodiments of the invention apply POPAR (patch order prediction and appearance recovery), a novel vision transformer-based self-supervised learning framework for chest X-ray images. POPAR leverages the benefits of vision transformers and unique properties of medical imaging, aiming to simultaneously learn patch-wise high-level contextual features by correcting shuffled patch orders and fine-grained features by recovering patch appearance. Embodiments of the invention transfer POPAR pre-trained models to diverse downstream tasks. Experimental results suggest that (1) POPAR outperforms self-supervised ImageNet models with transformer backbone; (2) POPAR outperforms SoTA self-supervised pretrained models with CNN and transformer backbones; and (3) POPAR also outperforms fully supervised pre-trained models across CNN and transformer architectures. In addition, an ablation study suggests that to achieve better performance on medical imaging tasks, both fine-grained and global contextual features are preferred.

POPAR is a vision transformer-based self-supervised learning method that supports both mainstream vision transformer architectures: The Vision Transformer (ViT) and Swin transformer. Generally speaking, an image transformer operates by dividing an image into fixed-size patches, correctly embedding each of the patches, and concatenating positional embedding as an input to a transformer encoder.

While the transformer architecture has become the de-facto standard for natural language processing tasks, its applications to computer vision are only now being realized. For the ViT, reliance on Convolutional Neural Networks (CNNs) is not mandatory and a pure transformer applied directly to sequences of image patches can perform very well on image classification tasks. When pre-trained on large amounts of data and transferred to multiple mid-sized or small image recognition benchmarks (e.g., such as using ImageNet, CIFAR-100, VTAB, etc.), the Vision Transformer (ViT) attains excellent results compared to state-of-the-art convolutional networks while requiring substantially fewer computational resources to train.

The Swin transformer can serve as a general-purpose backbone for computer vision. Challenges in adapting transformers from language to vision arose from differences between the two domains, such as large variations in the scale of visual entities and the high resolution of pixels in images compared to words in text. The Swin transformer addresses these differences using a hierarchical transformer whose representation is computed with shifted windows. The shifted windowing scheme brings greater efficiency by limiting self-attention computation to non-overlapping local windows while also allowing for cross-window connection. This hierarchical architecture has the flexibility to model at various scales and has linear computational complexity with respect to image size. These qualities of Swin transformers make them compatible with a broad range of vision tasks, including image classification.

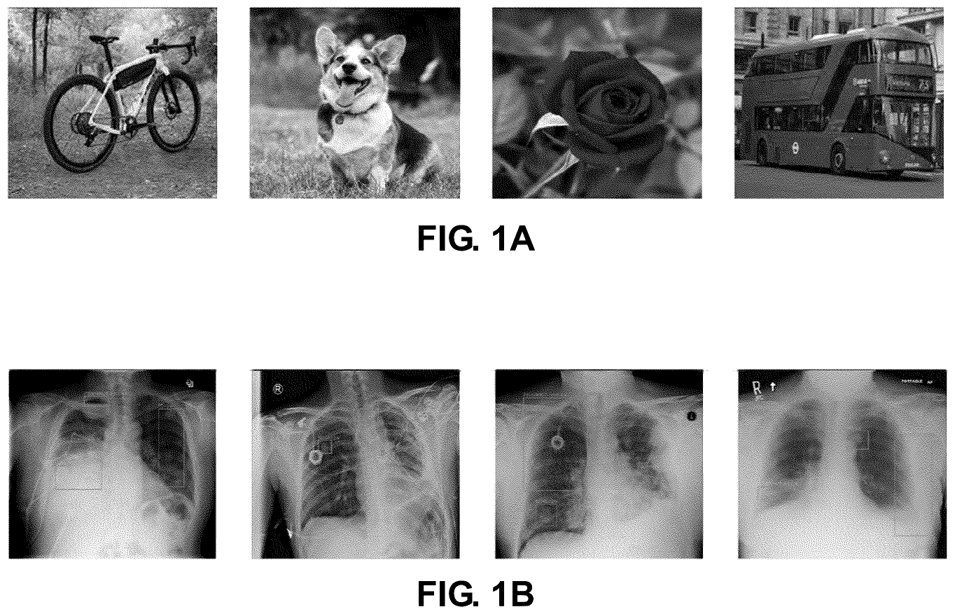

FIG. 1A depicts typical photographic images having objects of considerable differences on varying backgrounds compared with FIG. 1B which depicts medical images having similar appearances.

FIG. 1A provides exemplary photographic images that typically have objects of considerable differences (e.g., bicycle, dog, flower, etc.), each of which is centered in front of varying backgrounds, thus making the objects easily recognizable and less complex to segment the objects from the background data. Conversely, FIG. 1B provides exemplary medical images generated from an imaging protocol resulting in images that are remarkably similar in anatomy (e.g., lungs) across multiple distinct patients and with diagnostic information being spread across entire images (e.g., condition annotations).

Consequently, analyzing medical images requires not only high-level knowledge of anatomical structures and their relationships but also fine-grained features across entire images.

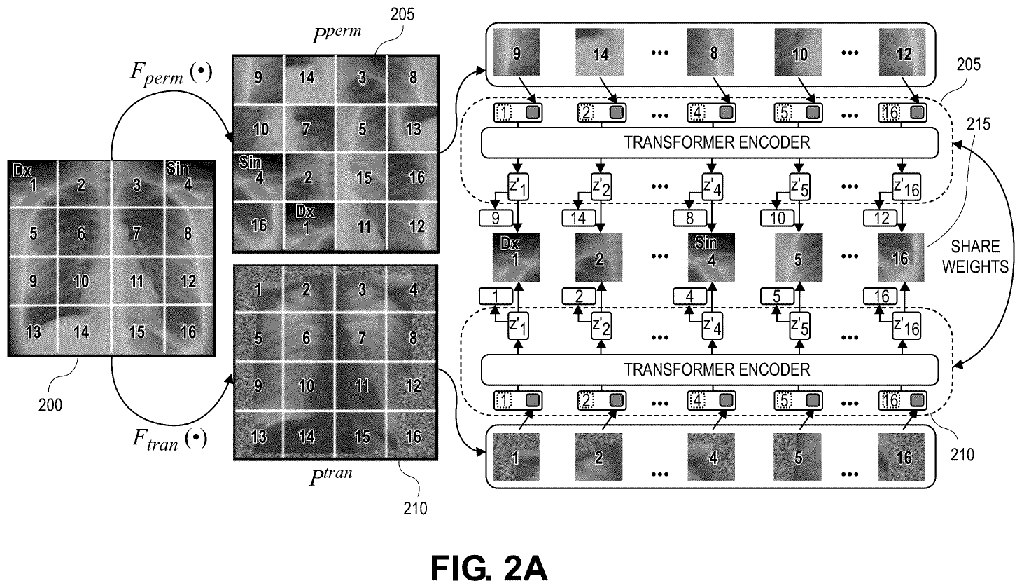

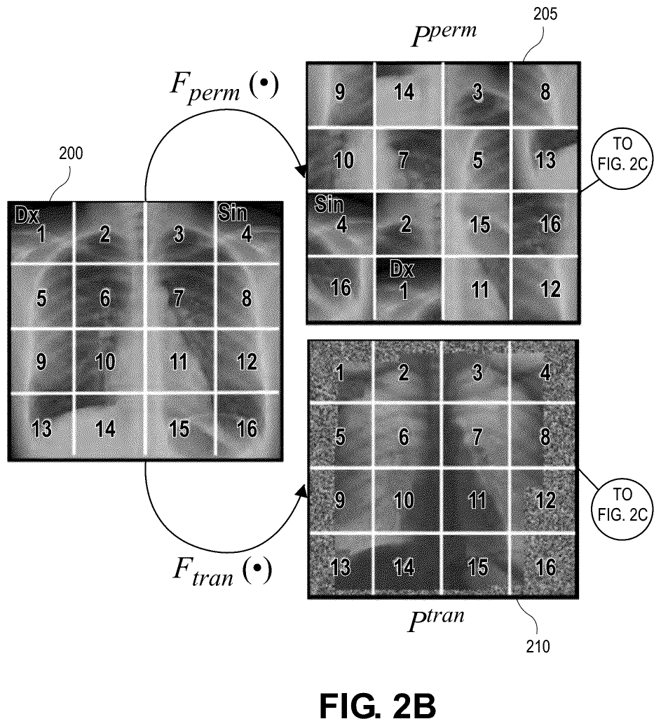

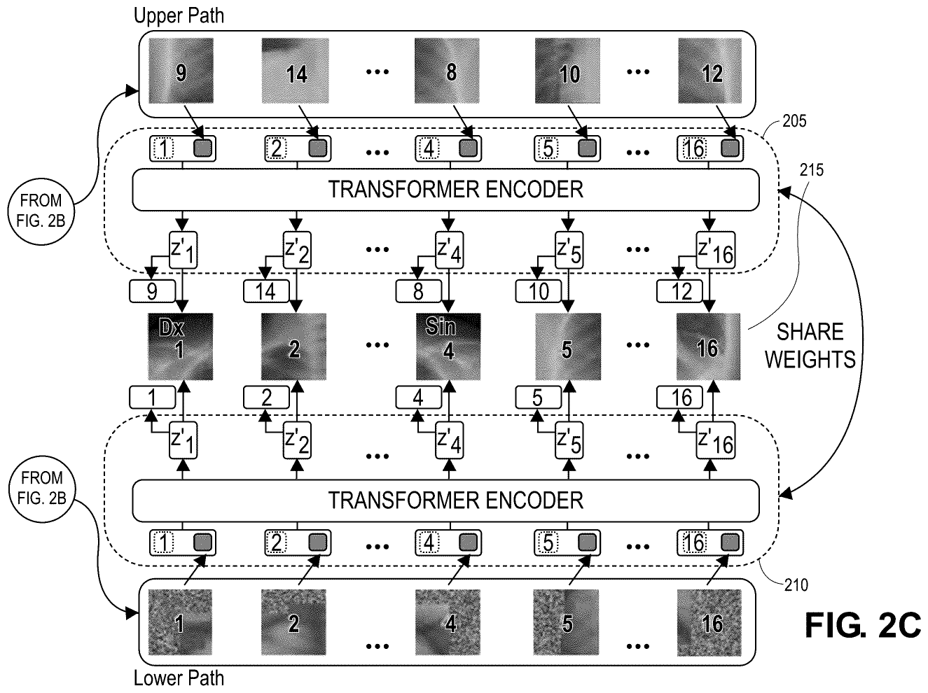

The POPAR methodology as described herein addresses these peculiar requirements by autodidactically learning high-level anatomical knowledge via patch order prediction and automatically gleaning fine-grained features via (patch) appearance recovery (Refer to FIG. 2A below).

### INTRODUCTION

Self-supervised learning (SSL) aims to learn generalizable representations from (unannotated) images and transfer the learned representations to application specific tasks to boost performance and reduce annotation efforts.

Self-supervised learning techniques are broadly considered to be the best available state-of-the-art techniques at this time, sometimes even surpassing standard supervised ImageNet models in computer vision. However, popularity of Self-supervised learning techniques within the field of medical imaging remains tepid, even in light of annotation dearth, thus presenting a significant challenge facing deep learning for Medical Image Analysis (MIA).

This lack of enthusiasm for broader adoption of self-supervised learning techniques may be due to the marked differences between medical and photographic images. As shown above with respect to FIG. 1A, typical non-medical photographic images, and particularly those utilized with the ImageNet model, typically have objects of considerable variations (cats, dogs, flowers, etc.), with distinctive components centered in front of varying backgrounds. Therefore, object recognition in photographic images is based mainly on high-level features extracted from objects' discriminative components.

Unfortunately, the same is not true in the context of Medical Image Analysis (MIA). Contrary to typical non-medical images, the development of medical imaging protocols has been designed for specified clinical purposes by focusing on particular body parts, consequently generating images of remarkable similarity in anatomy across patients.

For example, the posteroanterior chest X-rays as set forth at FIG. 1B, all look similar. And yet, diagnostically valuable information is often spread across entire images, and as such, an understanding of high-level anatomical structures and their relative spatial relationships is essential for distinguishing diseases from normal anatomy.

Notwithstanding the apparent similarities, the fine-grained details embedded throughout the entire expanse of the images are equally indispensable because identifying diseases, delineating organs, and isolating lesions may rely on subtle texture variations. Therefore, a natural question presented is: “How to learn integrated high-level and fine-grained features from medical images via self-supervision?”

To answer this question, a newly customized and specially configured self-supervised learning-based methodology is described herein, which is referred to as “POPAR” or “Patch Order Prediction and Appearance Recovery.” The novel POPAR methodology described herein is equipped with two novel learning perspectives. Specifically, (1) patch order prediction, which autodidactically learns high-level anatomical structures and their relative relationships, and (2) appearance recovery of patches, which automatically gleans fine-grained features from the medical images.

A Swin Transformer is utilized as the backbone for the POPAR methodology due to its hierarchical design which enables multi-scale modeling and thus naturally supports the two learning perspectives simultaneously.

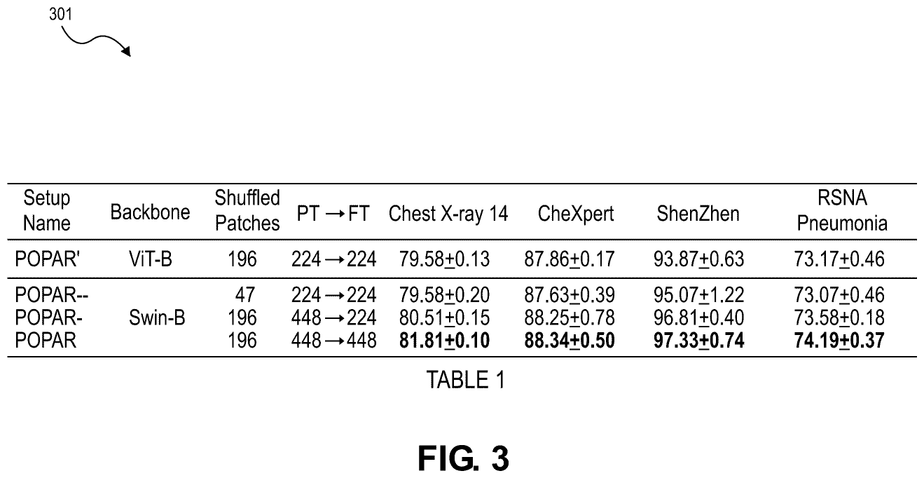

For performance comparison and ablation studies, three downgraded versions of the exemplary POPAR model were also trained, referred to herein as POPAR′ (POPAR prime), POPAR− (POPAR minus) and POPAR−− (POPAR minus minus), each of which is set forth below at Table 1 as presented at FIG. 3.

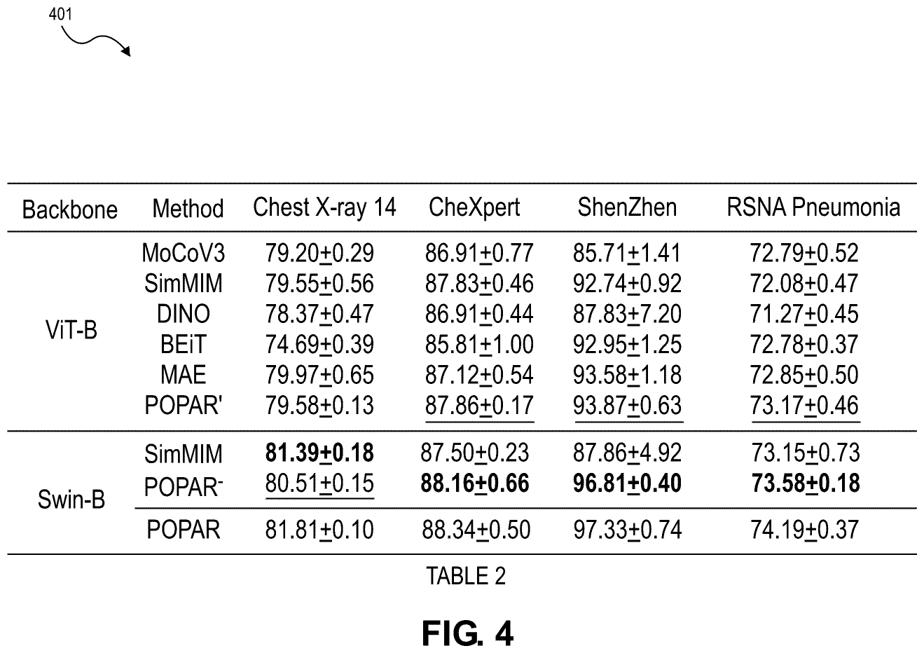

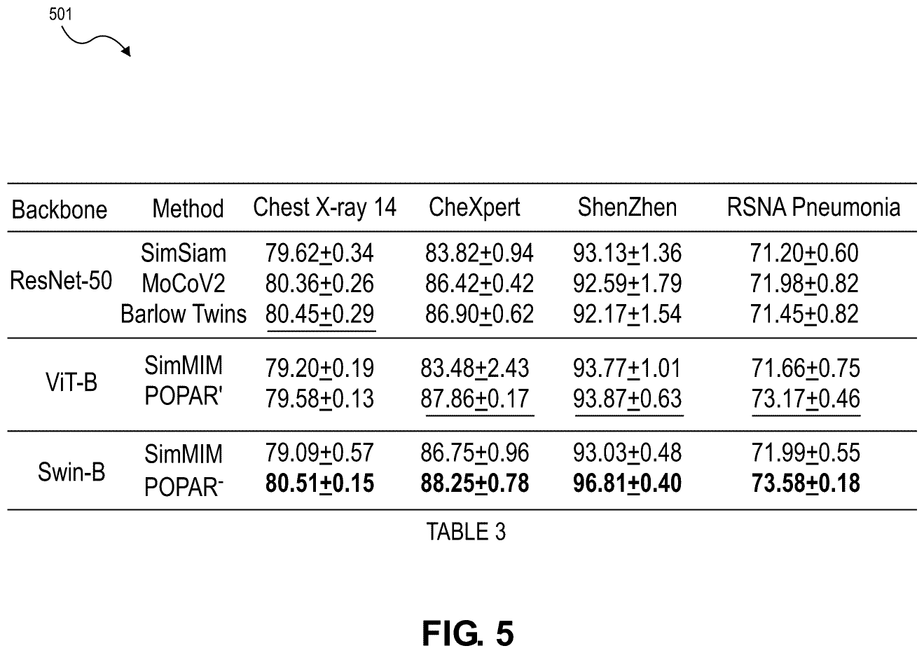

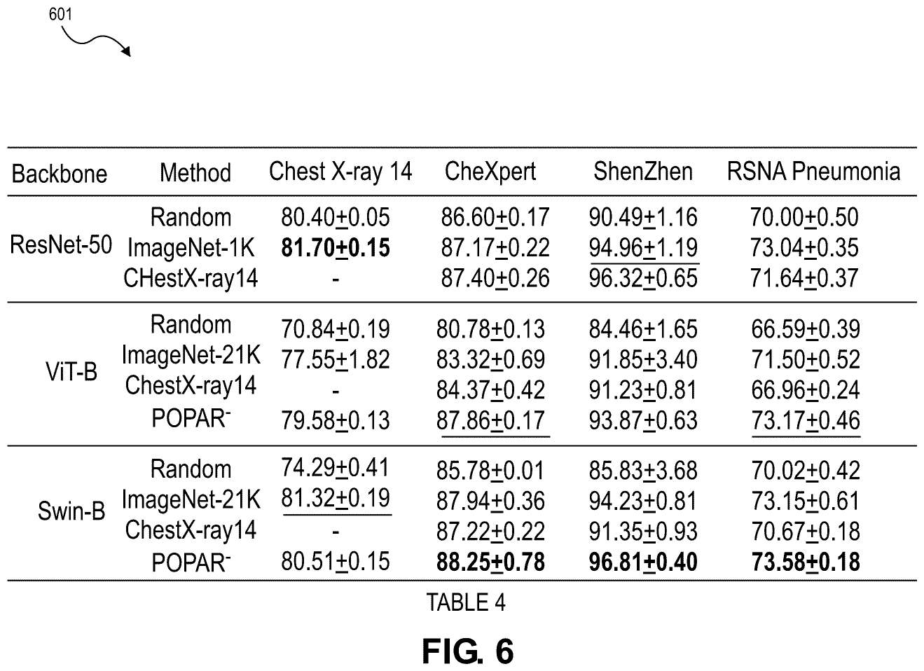

Through extensive experiments, it has been demonstrated that, firstly, (1) POPAR outperforms self-supervised ImageNet models with transformer backbone as shown by the results set forth below at Table 2 as presented at FIG. 4. Secondly, (2) POPAR outperforms other state-of-the-art self-supervised pre-trained models with CNN and transformer backbones as shown by the results set forth below at Table 3 as presented at FIG. 5. And thirdly, (3) POPAR outperforms fully supervised pre-trained models across CNN and transformer architectures as shown by the results set forth below at Table 4 as presented at FIG. 6.

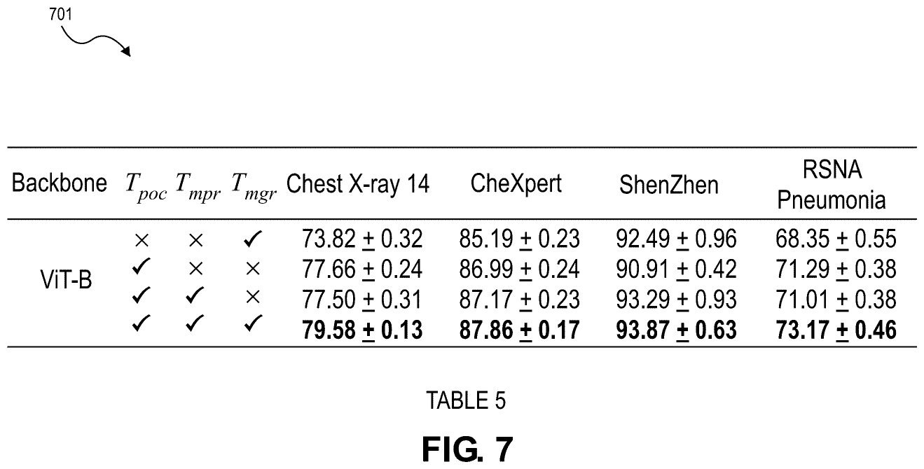

These performance improvements are attributable to insights into the requirements of medical imaging tasks for global anatomical knowledge and fine-grained details in texture variations. Refer to the results set forth below at Table 5 as presented at FIG. 7.

Consequently, the exemplary POPAR model described herein provides at least the following contributions: First, (1) a novel vision transformer-based SSL framework is provided that simultaneously learns global relationships of anatomical structures and fine-grained details embedded in medical images. Secondly, (2) a collection of pre-trained models for transformer architectures (ViT-B and Swin-B) are provided which yield state-of-the-art performance on a set of Medical Image Analysis (MIA) type classification tasks. And thirdly, (3) an extensive set of experiments demonstrate the POPAR model's superiority over other state-of-the-art supervised and self-supervised pre-trained models across varying architectures.

FIGS. 2A, 2B, and 2C depict an exemplary POPAR architecture, in accordance with described embodiments. More specifically, FIG. 2A shows the overall POPAR architecture, whereas FIGS. 2B and 2C show the same exemplary POPAR architecture as FIG. 2A, broken out into separate pages to display the architecture in greater detail.

The described POPAR methodology aims to learn (1) contextualized high-level anatomical structures via patch order prediction, and (2) fine-grained image features via patch appearance recovery. For each image, the image is first divided into a sequence of non-overlapping patches, for example, a sequence of sixteen patches as depicted at 200, and further processing randomly distorts the patch order via the upper path 205 or randomly distorts the patch appearances, that is, randomly distorting the unique portion of the medical image that appears in each patch, via the bottom path 210.

The distorted patch sequence is then provided to a transformer network, and the model is then trained to predict the correct position of each input patch and to also recover the correct patch appearance for each position as the original patch sequence, as depicted at 215.

Image context learning: Image context has been experimentally demonstrated to be a powerful source for learning visual representations via SSL. Multiple pretext tasks have been formulated to predict the context arrangement of image patches, including predicting the relative position of two image patches, specifically for solving Jigsaw puzzles and playing Rubik's cube.

Each of these methodologies employ multi-Siamese CNN backbones as feature extractors, followed by additional feature aggregation layers for determining the relationships between the input patches. However, the feature aggregation layers are discarded after the pre-training step, and only the pre-trained multi-Siamese CNNs are transferred to the target tasks. As a result, the learned relationships among image patches are mainly ignored in the target tasks.

Unlike prior approaches, the described POPAR methodology uses a multi-head attention mechanism to capture the relationships among anatomical patterns embedded in image patches, which is fully transferable to target tasks.

Masked image modeling: By customizing and extending upon prior masked language modeling techniques, various vision transformer-based SSL methodologies have proven beneficial for masked image modeling. For instance, the BEiT model predicts discrete tokens from masked images and the SimMIM and MAE models mask random patches from the input image and reconstruct the missing patches.

The disclosed POPAR methodologies adopts these broad strategies but then provides specialized customization and configuration specific to the context of processing medical imaging. Thus, the thus disclosed POPAR methodologies improve upon patch reconstruction and is distinguished from prior approaches by (1) reconstructing correct image patches from misplaced patches or from transformed patches, and (2) predicting the correct positions of shuffled image patches for learning global contextual features.

Restorative learning: The restorative SSL methods aim to learn representations by recovering original images from their distorted versions. For instance, Models Genesis has incorporated image restoration into their pretext tasks by using four effective image transformations for restorative SSL in medical imaging. The TransVW technique introduced an SSL framework for learning semantic representation from the consistent anatomical structures. The CAiD technique formulates a restoration task to boost instance discrimination SSL with context-aware representations. The DiRA methodology integrates discriminative, restorative, and adversarial SSL to learn fine-grained representations via collaborative learning.

However, none of these approaches can learn anatomical relationships among image patches. Conversely, the disclosed POPAR methodologies described herein employ a transformer backbone to integrate restorative learning with patch order prediction, capturing not only visual details but also relationships among anatomical structures.

Method:

Notations: Given an image sample x∈ [symbol] ^{H×W×C}, where (H, W) is the resolution of the image and C is the number of channels, one of the following distortion functions are selected and applied: (a) patch order distortion Fₚₑᵣₘ(·) which corresponds to the upper path 205 as shown at FIGS. 2A, 2B, and 2C or alternatively, (b) patch appearance distortion Fₜᵣₐₙ(·) which corresponds to the lower path 210 as shown at FIGS. 2A, 2B, and 2C.

To apply patch order distortion, x is first divided into a sequence of n non-overlapping image patches P=(p₁, p₂, . . . , pₙ), where

$$n = \frac{HxW}{{k}^{2}}$$

and (k, k) is the resolution of each patch. The term L=(1, 2, . . . , n) is used to denote the correct patch positions within x. A random permutation operator is then applied on L to generate the permuted patch positions L^{perm}. Next, L^{perm} is used to re-arrange the patch sequence P, resulting in permuted patch sequence P^{perm}.

To apply patch appearance distortion, an image transformation operator is first applied on x, resulting in an appearance-transformed image x^{tran}. Next, x^{tran} is divided into a sequence of n non-overlapping transformed image patches P^{tran}=(p₁^{tran}, p₂^{tran}, . . . , pₙ^{tran}). Next the patches are mapped in P^{perm} and P^{tran} into D dimension patch embeddings using a trainable linear projection layer.

The patch appearance distortion processing then continues by adding trainable positional embeddings to the patch embeddings, resulting in a sequence of embedding vectors. The embedding vectors are further processed by the transformer encoder g_{θ}(·) to generate a set of contextual patch features Z′=(z′₁, z′₂, . . . , z′ₙ). Next, Z′ is then passed onto two distinct prediction heads s_{θ}(·) and k_{θ}(·) to generate predictions p^{pop}=s_{θ}(Z′) and p^{ar}=p^{ar}=k_{θ}(Z′) for performing the patch order prediction and patch appearance recovery, respectively, as described below. Lastly, ! is defined as “shall be (made) equal.”

Patch order prediction aims to predict the correct position of a patch based on its appearance. Particularly, depending on which distortion function is selected, the expected prediction for p^{pop} is formulated in accordance with equation 1, as follows:

$$\{ \begin{matrix}{𝒫}^{pop} {=}^{!} {L}_{perm} & If {ℱ}_{perm} ( \cdot ) is selected \\ {𝒫}^{pop} {=}^{!} L & If {ℱ}_{tran} ( \cdot ) is selected\end{matrix} .$$

Patch appearance recovery aims to reconstruct the correct appearance for each position in the input sequence. The network is expected to predict the original appearance in P regardless of which distortion function (Fₚₑᵣₘ(·) or Fₜᵣₐₙ(·) is selected. The expected reconstruction prediction for p^{ar} is defined in accordance with equation 2, as follows:

$$𝒫 {ar}^{} {=}^{!} P .$$

Overall training scheme: The patch order prediction is formulated as an n-way multi-class classification task and the model is optimized by minimizing the categorical cross-entropy loss:

$${ℒ}_{pop} = - \frac{1}{B} {\sum }_{b = 1}^{B} {\sum }_{l = 1}^{n} {\sum }_{c = 1}^{n} {y}_{blc} log {𝒫}_{blc}^{pop} ,$$

where B denotes the batch size, n is the number of patches for each image, Y represents the ground truth (as defined above at equation 1), and where p^{pop} represents the network's patch order prediction.

The patch appearance recovery is formulated as a reconstruction task and the model is trained by minimizing the L2 distance between the original patch sequence P and the restored patch sequence p^{ar}:

$${ℒ}_{ar} = \frac{1}{B} {\sum }_{b = 1}^{B} {\sum }_{j = 1}^{n} {‖ {p}_{j} - {p}_{j}^{ar} ‖}_{2}^{2} ,$$

where pⱼ and pⱼ^{ar} represent the patch appearance from P and p^{ar}, respectively.

Both learning schemes are then integrated and POPAR is then trained with an overall loss function Lₚₒₚₐᵣ=λ*Lₚₒₚ+(1−λ)*Lₐᵣ, where λ is the weight to specify the importance of each loss. The formulation of the Lₚₒₚ encourages the transformer model to learn high-level anatomical structures and their relative relationships. Moreover, the definition of Lₐᵣ encourages the model to capture more fine-grained features from images.

Experiments:

Implementation Details and Pre-training settings: To start, the POPAR model was pre-trained using ViT-B and Swin-B as backbones using their default configurations on the training set of the ChestXray14 dataset. Due to architectural differences, an image size of 224×224 and 448×448 was utilized for each of the ViT-B and Swin-B backbones, respectively.

The images were therefore divided into 16×16 and 32×32 patches for ViT-B and Swin-B, respectively, which resulted in n=196 patches in both backbones.

Two single linear layers were then utilized as the prediction heads for the classification (order prediction) and restoration (appearance recovery) tasks. For all models, the SGD optimizer was used with a learning rate 0.1 and λ was set to 0.5.

The POPAR models were trained with ViT-B and Swin-B backbones for 1000 and 300 epochs respectively. The image transformation function Fₜᵣₐₙ(·) includes local pixel shuffling, non-linear transformation, and outer/inner cutouts.

Target tasks and fine-tuning settings: The efficacy of the POPAR models were then evaluated in transfer learning to four medical classification tasks in chest X-ray datasets including ChestX-ray14, CheXpert, NIH Shenzhen CXR, and RSNA Pneumonia.

The POPAR models were transferred to target tasks by removing the prediction heads and inserting randomly initialized target classification heads that include (1) a linear layer for the ViT-B backbone and (2) an average pooling and a linear layer for the Swin-B backbone and all the parameters of the target models were fine-tuned.

FIG. 3 depicts Table 1 (element 301) which shows an evaluation of POPAR with ViT-B and Swin-B backbones, in accordance with described embodiments. In particular, the POPAR models were evaluated with ViT-B and Swin-B backbones using four different pre-training and fine-tuning image resolutions, denoted as PT and FT, respectively. The POPAR model with the ViT-B backbone as pre-trained and fine-tuned on 224 resolution, denoted as “POPAR-prime” or POPAR′. The POPAR model with the Swin-B backbone and pre-training and fine-tuning resolution of 224 is denoted as “POPAR-minus-minus” or POPAR−−. The POPAR model with Swin-B backbone, pre-training size of 448, and fine-tuning size of 224 is denoted as “POPAR-minus” or POPAR−. Finally, the model with Swin-B backbone, pre-training and fine-tuning size of 448 is denoted as simply “POPAR,” which is shown experimentally to yield the best performance on all target tasks and corresponds to the implementation as described herein.

FIG. 4 depicts Table 2 (element 401) which shows that POPAR models experimentally outperform other known state-of-the-art self-supervised ImageNet models with transformer backbone in three target tasks, in accordance with described embodiments. The best methods are bolded, while the second best are underlined.

FIG. 5 depicts Table 3 (element 501) which shows that POPAR models yield significant performance boosts over other known state-of-the-art techniques, in accordance with described embodiments. In particular, the POPAR models described herein were experimentally shown to yield significant performance boosts (p<0.05) in comparison with other known state-of-the-art self-supervised methods pre-trained on ResNet-50 or transformer architectures. All models were pre-trained on the ChestX-ray14 dataset. The best methods are bolded while the second best are underlined.

FIG. 6 depicts Table 4 (element 601) which shows that POPAR models were experimentally shown to outperform fully supervised pre-trained models on ImageNet and ChestX-ray14 datasets in three target tasks across architectures, in accordance with described embodiments. The best methods are bolded while the second best are underlined. Note that transfer learning is inapplicable when pre-training and target tasks are the same, which is denoted in the table by the symbol “−”.

FIG. 7 depicts Table 5 (element 701) which shows how each component in POPAR is used, in accordance with described embodiments. The performance increases gradually by adding subtasks. As shown here, T_{poc} represents shuffled patch order classification only, Tₘₚᵣ represents misplaced patch restoration only, and T_{mgr} represents the Models Genesis transformed image restoration only. For MIA tasks it is demonstrated that all subtasks are used to obtain both fine-grained and global contextual features, resulting in a better performance.

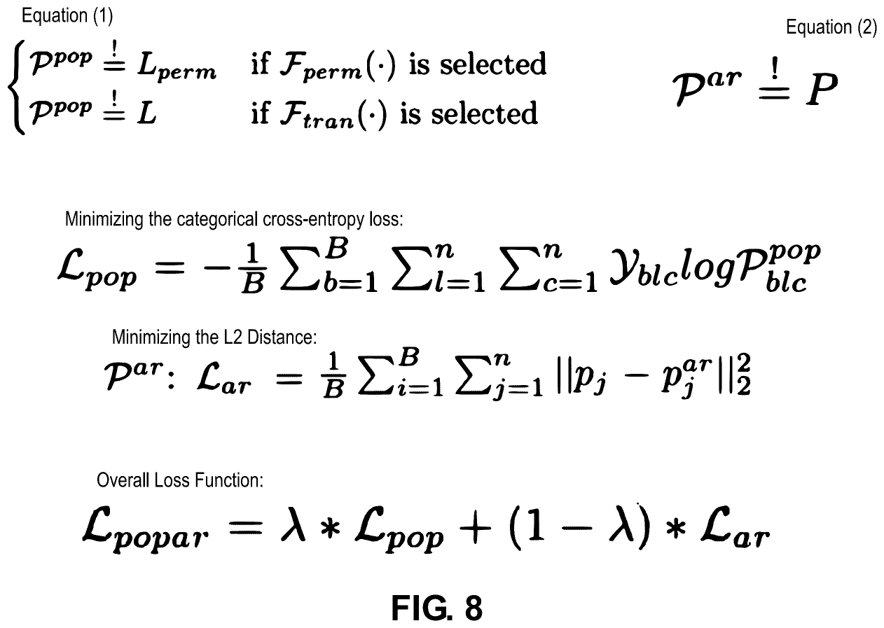

FIG. 8 depicts equations as utilized in conjunction with the POPAR framework and trained POPAR models, in accordance with described embodiments.

Results:

For a first experiment, POPAR outperformed self-supervised ImageNet models with transformer backbone. To demonstrate the effectiveness of pre-training transformers with in-domain medical data, POPAR was compared with state-of-the-art transformer-based self-supervised methods that were pre-trained on ImageNet.

Existing self-supervised ImageNet models were evaluated with ViT-B (e.g., MoCoV3, SimMIM, DINO, BEiT, and MAE) as well as Swin-B (e.g., SimMIM) backbones. The officially released models for all baselines were utilized among which the BEiT model was pre-trained on the ImageNet-21K dataset, while the rest of the models were pre-trained on the ImageNet-1K dataset.

From experimental results as set forth at Table 2 (presented at FIG. 4), the following observations are made. Firstly, SimMIM and MAE achieve superior performance over other baselines, demonstrating the effectiveness of masked image restoration for pre-training transformer models. Secondly, POPAR with ViT-B backbone surpasses all self-supervised ImageNet models with the same backbone. Thirdly, POPAR outperforms SimMIM with Swin-B backbone on three out of four target tasks.

For a second experiment, POPAR outperformed self-supervised pre-trained models across architectures. To demonstrate the effectiveness of representation learning via the framework described herein, POPAR was compared with other state-of-the-art CNN-based and transformer-based SSL methods having been pre-trained on medical images.

Specifically, three recent SSL methods were (1) first evaluated with the ResNet-50 backbone, including MoCoV2, Barlow Twins, and SimSiam, and (2) secondly evaluated with SimMIM, which has shown superior performance over other transformer-based SSL methods in both vision and medical tasks, with ViT-B and Swin-B backbones (refer again to Table 2 as presented at FIG. 4). All models were pre-trained on ChestX-ray14 dataset.

With reference to Table 3 as presented at FIG. 5, POPAR was experimentally shown to yield significantly better performance when compared with three SSL methods with ResNet-50 backbone in all target tasks. Moreover, POPAR model was shown to outperform SimMIM in all target tasks across ViT-B and Swin-B backbones. These results demonstrate that POPAR models provide more useful representations for various medical imaging tasks.

For a third experiment, POPAR outperformed fully supervised pre-trained models across architectures. The POPAR models compared were pre-trained on unlabeled images of ChestX-ray14 dataset, with fully supervised pre-trained models on ImageNet and ChestX-ray14 across three architectures: ResNet-50, ViT-B, and Swin-B. Existing supervised ImageNet models were utilized with CNN and transformer backbones pre-trained on ImageNet-1K and ImageNet-21K datasets, respectively.

With reference to Table 4 as presented at FIG. 6, POPAR was experimentally shown to yield superior performance over both supervised ImageNet and ChestX-ray14 models across architectures in three target tasks. In particular, POPAR models with ViT-B and Swin-B backbones outperform corresponding supervised baselines with the same backbone in all and three target tasks, respectively. Moreover, POPAR with Swin-B backbone outperformed supervised models with ResNet-50 backbone in three target tasks.

The above experimental results demonstrate that POPAR provides more generic features for various medical imaging tasks.

Ablation Study Impact of input resolutions: Further evaluations of POPAR were conducted with ViT-B and SwinB backbones using four different pre-training and fine-tuning image resolutions.

With reference to Table 1 as presented at FIG. 3, compared with POPAR⁻⁻ and POPAR⁻, a larger number of shufflable patches provides a larger performance gain on all target tasks. Moreover, with the same number of shufflable patches, POPAR− with a Swin-B backbone provides superior performance compared with POPAR′ with a ViTB backbone. Consequently, the Swin transformer is the most suggested POPAR backbone.

Lastly, POPAR was pre-trained and fine-tuned with 448×448 resolution, denoted by POPAR (refer again to Table 1 as presented at FIG. 3), suggests the state-of-the-art performance on all four target tasks. It indicates that the higher input resolution is preferred for all four MIA tasks studied evaluated, since higher resolution provides more detailed anatomical information, thus enhancing the performance of all MIA target tasks.

Pre-training tasks: When performing pre-training, POPAR seamlessly combines two tasks: patch order prediction and patch appearance recovery. With reference to Table 5 as presented at FIG. 7, the tasks can be further broken down into three individual sub-tasks: (a) patch order classification, denoted by T_{poc}; (b) misplaced patch appearance recovery, denoted by Tₘₚᵣ; and (c) Models Genesis transformed image restoration, denoted by T_{mgr}.

The effectiveness of different POPAR pre-training subtasks were evaluated on the ViT-B backbone. As shown in Table 5, compared with the Models Genesis transformed image restoration, the patch order prediction task provides a significant performance boost on most target tasks. Furthermore, the combination of the misplaced patch appearance recovery task and the patch order classification task provides an on-par or less performance increment on four target tasks (see the third row in Table 5). Thus, it was experimentally demonstrated that POPAR pre-trained with all subtasks provides the highest performance boost.

Implementation Details: POPAR can be generalized easily on the vision transformer architectures. POPAR may be implemented on ViT-Base (ViT-B) and Swin-Base (Swin-B) models as they are more computationally feasible, and the most selected architectures by the recent vision transformer-based self-supervised learning methods.

POPAR may be pre-trained on ViT-B and Swin-B based on their official default configurations with the NIH ChestX-ray14 official training data split. Both take 224×224 as their input image size, resulting in 196 (14×14) shufflable patches for ViT-B. Due to the hierarchical structure of the Swin Transformer, the resulting shufflable patches are 49 (7×7) for Swin-B.

To learn the same contextual relationship as the ViT-B, POPAR may be pre-trained on Swin-B with 448×448 input image size, while the tissue (physical) size remains unchanged, resulting in the same 196 (14×14) shufflable patches.

One limitation of CNN architecture is that it utilizes the up-sampling layer followed by a series of convolutions blocks to recover an image, whereas the vision transformer can use a single linear layer to accomplish the recovery. Consequently, a 196 multi-class patch order classification task may be formed for ViT-B with 224×224, and Swin-B with 448×448 input image size. For Swin-B with 224×224 input image size, the number of patch order class is 49 because of its hierarchical structure. The learning rate is set to 0.1 with a warm-up of 5 epochs and 0.5 weight decay.

Four Nvidia Telsa V100 32 GB GPUs may be utilized for training the POPAR models with an image size of 224×224 for 1000 epochs, but the number of epochs may be reduced to 300 when training POPAR models with an image size of 448×448 due to the long training time caused by the larger image size.

Target Tasks and Datasets: The POPAR models may be fine-tuned to four classification target tasks: Firstly, (1) NIH ChestX-ray14, which contains 112K frontal view chest X-ray images; in which each image is associated with 14 labels for thoracic diseases, and in which the official training (86K), and testing (25K) splits were used; Secondly, (2) CheXpert, including 224K frontal-view chest X-ray images may be used and similar to NIH ChestX-ray14, each image is labeled with 14 thoracic diseases, in which the official data split is again utilized, including 224K training images and 234 test images; Thirdly, (3) Shenzhen CXR may be used which consists of 326 normal and 336 Tuberculosis (TB) frontal-view chest X-ray images; and fourthly, (4) RSNA Pneumonia classification may be used which contains 30K frontal view chest X-ray images.

Each image is associated with a distinct diagnosis label, such as Normal, Lung Opacity (Pneumonia), or Not Normal (other diseases). These target datasets are composed of both multi-label and multi-class classification tasks with various diseases. Furthermore, these tasks contain many typical obstacles when working with medical images, such as data imbalance and data scarcity. If official training and testing splits are not available, data samples may be chosen at random with 80% and 20% for training and testing, respectively.

Fine-tuning Settings: The POPAR pre-trained models may be transferred to each target task by fine-tuning all the parameters of the target task models. To prevent the issue of over-fitting, 10% of the training set may be utilized as the validation set for early stopping. To obtain the final classification feature, a randomly initialized linear layer may be concatenated to the output of the classification (CLS) token of POPAR ViT-B models. Due to the structural differences, POPAR Swin-B models are not able to inject CLS token; as a result, the classification feature vector may be obtained by performing an average pooling operation on the last feature map, and then by feeding the feature to the randomly initialized linear layer.

The AUC (area under the ROC curve) may be used to assess multi-label classification performance (NIH ChestX-ray14, CheXpert, and Shenzhen CXR), whereas the Accuracy is used to evaluate RSNA Pneumonia multiclass classification performance. For each target task, the mean performance, standard deviation, and statistical analysis may be reported based on ten independent runs. Refer again to the ablation study results as set forth at Table 5.

### Definitions

The articles “a” and “an” are used in this disclosure to refer to one or more than one (i.e., to at least one) of the grammatical object of the article. By way of example, “a medical image” means one medical image or more than one medical image.

The term “and/or” is used in this disclosure to mean either “and” or “or” unless indicated otherwise.

### ENUMERATED EMBODIMENTS

Embodiment 1: A method comprising:
receiving a plurality of medical images;
dividing each of the plurality of medical images into a sequence of image patches;
shuffling the sequence of image patches for each of the plurality of medical images;
transforming each of the sequence of image patches for each of the plurality of medical images;
integrating instructions for performing operations for both:
   (1) reconstructing image patches from the transformed image patches for each the plurality of medical images; and
   (2) predicting corrected positions of the shuffled sequence of image patches in the plurality of medical images for learning global contextual features;
wherein reconstructing image patches comprises applying restorative Self-Supervised-Learning operations to learn representations by recovering the plurality of medical images from the transformed image patches; and
wherein predicting corrected positions of the shuffled sequence of image patches comprises applying patch order prediction to capture both visual details and associated relationships among anatomical structures for the plurality of medical images as represented within the shuffled sequence of image patches.

Embodiment 2: A self-supervised machine learning method for learning visual representations in medical images, comprising:
receiving a plurality of medical images of similar anatomy;
dividing each of the plurality of medical images into its own sequence of non-overlapping patches, wherein a unique portion of each medical image appears in each patch in the sequence of non-overlapping patches;
randomizing the sequence of non-overlapping patches for each of the plurality of medical images;
randomly distorting the unique portion of each medical image that appears in each patch in the sequence of non-overlapping patches for each of the plurality of medical images;
learning, via a vision transformer network, patch-wise high-level contextual features in the plurality of medical images; and
learning simultaneously, via the vision transformer network, fine-grained features embedded in the plurality of medical images.

Embodiment 3: The method of embodiment 2, wherein learning, via a vision transformer network, patch-wise high-level contextual features comprises learning high-level anatomical structures and their relative relationships in the plurality of medical images.

Embodiment 4: The method of any one of embodiments 2 or 3, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

Embodiment 5: The method of any one of embodiments 2-4, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

Embodiment 6: The method of any one of embodiments 2-5, wherein learning simultaneously, via the vision transformer network, fine-grained features embedded in the plurality of medical images comprises learning details in texture variations embedded throughout an entirety of the plurality of medical images.

Embodiment 7: The method of any one of embodiments 2-6, wherein learning simultaneously, via the vision transformer network, fine-grained features embedded in the plurality of medical images comprises:
providing the randomly distorted unique portion of each medical image that appears in each patch in the sequence of non-overlapping patches for each of the plurality of medical images to the vision transformer network; and
training the vision transformer network to recover the unique portion of each medical image that appears in each patch in the sequence of non-overlapping patches for each of the plurality of medical images.

Embodiment 8: A method comprising:
receiving a medical image;
dividing each medical image into a sequence of image patches;
shuffling the sequence of image patches for each medical image;
transforming each of the sequence of image patches for each medical image;
integrating instructions for performing operations for both:
   (1) reconstructing image patches from the transformed image patches for each medical image; and
   (2) predicting corrected positions of the shuffled sequence of image patches in the medical image for learning global contextual features;
wherein reconstructing image patches comprises applying restorative Self-Supervised-Learning operations to learn representations by recovering the medical image from the transformed image patches; and
wherein predicting corrected positions of the shuffled sequence of image patches comprises applying patch order prediction to capture both visual details and associated relationships among anatomical structures for the medical image as represented within the shuffled sequence of image patches.

Embodiment 9: A system comprising:
a memory to store instructions; and
a processor to execute the instructions stored in the memory;
   wherein the system is specially configured to execute instructions via the processor for performing the following operations:
receiving a plurality of medical images of similar anatomy;
dividing each of the plurality of medical images into its own sequence of non-overlapping patches, wherein a unique portion of each medical image appears in each patch in the sequence of non-overlapping patches;
randomizing the sequence of non-overlapping patches for each of the plurality of medical images;
randomly distorting the unique portion of each medical image that appears in each patch in the sequence of non-overlapping patches for each of the plurality of medical images;
learning, via a vision transformer network, patch-wise high-level contextual features in the plurality of medical images; and
learning simultaneously, via the vision transformer network, fine-grained features embedded in the plurality of medical images.

Embodiment 10: The system of embodiment 9, wherein learning, via a vision transformer network, patch-wise high-level contextual features comprises learning high-level anatomical structures and their relative relationships in the plurality of medical images.

Embodiment 11: The system of any one of embodiments 9-10, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

Embodiment 12: The system of any one of embodiments 9-11, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

Embodiment 13: The system of any one of embodiments 9-12, wherein learning simultaneously, via the vision transformer network, fine-grained features embedded in the plurality of medical images comprises learning details in texture variations embedded throughout an entirety of the plurality of medical images.

Embodiment 14: The system of any one of embodiments 9-13, wherein learning simultaneously, via the vision transformer network, fine-grained features embedded in the plurality of medical images comprises:
providing the randomly distorted unique portion of each medical image that appears in each patch in the sequence of non-overlapping patches for each of the plurality of medical images to the vision transformer network; and
training the vision transformer network to recover the unique portion of each medical image that appears in each patch in the sequence of non-overlapping patches for each of the plurality of medical images.

Embodiment 15: A non-transitory computer readable storage media having instructions stored thereupon that, when executed by a process of a system specially configured for diagnosing disease within new medical images;
wherein the instructions cause the system to perform operations including:
   receiving a plurality of medical images;
   receiving a plurality of medical images of similar anatomy;
   dividing each of the plurality of medical images into its own sequence of non-overlapping patches, wherein a unique portion of each medical image appears in each patch in the sequence of non-overlapping patches;
   randomizing the sequence of non-overlapping patches for each of the plurality of medical images;
   randomly distorting the unique portion of each medical image that appears in each patch in the sequence of non-overlapping patches for each of the plurality of medical images;
   learning, via a vision transformer network, patch-wise high-level contextual features in the plurality of medical images; and
   learning simultaneously, via the vision transformer network, fine-grained features embedded in the plurality of medical images.

Embodiment 16: The non-transitory computer readable storage media of embodiment 15, wherein learning, via a vision transformer network, patch-wise high-level contextual features comprises learning high-level anatomical structures and their relative relationships in the plurality of medical images.

Embodiment 17: The non-transitory computer readable storage media of embodiments 15 or 16, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

Embodiment 18: The non-transitory computer readable storage media of any one of embodiments 15-17, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

Embodiment 19: The non-transitory computer readable storage media of any one of embodiments 15-18, wherein learning simultaneously, via the vision transformer network, fine-grained features embedded in the plurality of medical images comprises learning details in texture variations embedded throughout an entirety of the plurality of medical images.

Embodiment 20: The non-transitory computer readable storage media of any one of embodiments 15-19, wherein learning simultaneously, via the vision transformer network, fine-grained features embedded in the plurality of medical images comprises: providing the randomly distorted unique portion of each medical image that appears in each patch in the sequence of non-overlapping patches for each of the plurality of medical images to the vision transformer network; and training the vision transformer network to recover the unique portion of each medical image that appears in each patch in the sequence of non-overlapping patches for each of the plurality of medical images.

Concluding Remarks:

The POPAR methodologies, models, and framework as described herein is therefore presented as a novel transformer-based SSL framework for MIA tasks. POPAR integrates patch order prediction and appearance recovery, capturing not only high-level relationships among anatomical structures but also fine-grained details from medical images.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A method comprising:
receiving a plurality of medical images;
dividing each of the plurality of medical images into a sequence of image patches;
shuffling the sequence of image patches for each of the plurality of medical images;
transforming each of the sequence of image patches for each of the plurality of medical images;
integrating instructions for performing operations for both:
(1) reconstructing image patches from the transformed image patches for each the plurality of medical images; and
(2) predicting corrected positions of the shuffled sequence of image patches in the plurality of medical images for learning global contextual features;
wherein reconstructing image patches comprises applying restorative Self-Supervised-Learning operations to learn representations by recovering the plurality of medical images from the transformed image patches; and
wherein predicting corrected positions of the shuffled sequence of image patches comprises applying patch order prediction to capture both visual details and associated relationships among anatomical structures for the plurality of medical images as represented within the shuffled sequence of image patches.

2. A self-supervised machine learning method for learning visual representations in medical images, comprising:
receiving a plurality of medical images of similar anatomy;
dividing each of the plurality of medical images into its own sequence of non-overlapping patches, wherein a unique portion of each medical image appears in each patch in the sequence of non-overlapping patches;
randomizing the sequence of non-overlapping patches for each of the plurality of medical images;
randomly distorting the unique portion of each medical image that appears in each patch in the sequence of non-overlapping patches for each of the plurality of medical images;
learning, via a vision transformer network, patch-wise high-level contextual features in the plurality of medical images; and
learning simultaneously, via the vision transformer network, fine-grained features embedded in the plurality of medical images.

3. The method of claim 2, wherein learning, via a vision transformer network, patch-wise high-level contextual features comprises learning high-level anatomical structures and their relative relationships in the plurality of medical images.

4. The method of claim 2, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

5. The method of claim 4, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

6. The method of claim 2, wherein learning simultaneously, via the vision transformer network, fine-grained features embedded in the plurality of medical images comprises learning details in texture variations embedded throughout an entirety of the plurality of medical images.

7. The method of claim 2, wherein learning simultaneously, via the vision transformer network, fine-grained features embedded in the plurality of medical images comprises:
providing the randomly distorted unique portion of each medical image that appears in each patch in the sequence of non-overlapping patches for each of the plurality of medical images to the vision transformer network; and
training the vision transformer network to recover the unique portion of each medical image that appears in each patch in the sequence of non-overlapping patches for each of the plurality of medical images.

8. A system comprising:
a memory to store instructions; and
a processor to execute the instructions stored in the memory;
wherein the system is specially configured to execute instructions via the processor for performing the following operations:
receiving a plurality of medical images of similar anatomy;
dividing each of the plurality of medical images into its own sequence of non-overlapping patches, wherein a unique portion of each medical image appears in each patch in the sequence of non-overlapping patches;
randomizing the sequence of non-overlapping patches for each of the plurality of medical images;
randomly distorting the unique portion of each medical image that appears in each patch in the sequence of non-overlapping patches for each of the plurality of medical images;
learning, via a vision transformer network, patch-wise high-level contextual features in the plurality of medical images; and
learning simultaneously, via the vision transformer network, fine-grained features embedded in the plurality of medical images.

9. The system of claim 8, wherein learning, via a vision transformer network, patch-wise high-level contextual features comprises learning high-level anatomical structures and their relative relationships in the plurality of medical images.

10. The system of claim 8, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

11. The system of claim 10, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

12. The system of claim 8, wherein learning simultaneously, via the vision transformer network, fine-grained features embedded in the plurality of medical images comprises learning details in texture variations embedded throughout an entirety of the plurality of medical images.

13. The system of claim 8, wherein learning simultaneously, via the vision transformer network, fine-grained features embedded in the plurality of medical images comprises:
providing the randomly distorted unique portion of each medical image that appears in each patch in the sequence of non-overlapping patches for each of the plurality of medical images to the vision transformer network; and
training the vision transformer network to recover the unique portion of each medical image that appears in each patch in the sequence of non-overlapping patches for each of the plurality of medical images.

14. A non-transitory computer readable storage media having instructions stored thereupon that, when executed by a process of a system specially configured for diagnosing disease within new medical images;
wherein the instructions cause the system to perform operations including:
receiving a plurality of medical images;
receiving a plurality of medical images of similar anatomy;
dividing each of the plurality of medical images into its own sequence of non-overlapping patches, wherein a unique portion of each medical image appears in each patch in the sequence of non-overlapping patches;
randomizing the sequence of non-overlapping patches for each of the plurality of medical images;
randomly distorting the unique portion of each medical image that appears in each patch in the sequence of non-overlapping patches for each of the plurality of medical images;
learning, via a vision transformer network, patch-wise high-level contextual features in the plurality of medical images; and
learning simultaneously, via the vision transformer network, fine-grained features embedded in the plurality of medical images.

15. The non-transitory computer readable storage media of claim 14, wherein learning, via a vision transformer network, patch-wise high-level contextual features comprises learning high-level anatomical structures and their relative relationships in the plurality of medical images.

16. The non-transitory computer readable storage media of claim 14, wherein learning, via the vision transformer network, patch-wise high-level contextual features in the plurality of medical images comprises:
providing the randomized sequence of non-overlapping patches for each of the plurality of medical images to the vision transformer network; and
training the vision transformer network to predict the sequence of non-overlapping patches for each of the plurality of medical images.

17. The non-transitory computer readable storage media of claim 16, wherein training the vision transformer network to predict the sequence of non-overlapping patches for each of the plurality of medical images comprises training the vision transformer network to predict the sequence of non-overlapping patches for each of the plurality of medical images based on an appearance of each patch in the sequence of non-overlapping patches for each of the plurality of medical images.

18. The non-transitory computer readable storage media of claim 14, wherein learning simultaneously, via the vision transformer network, fine-grained features embedded in the plurality of medical images comprises learning details in texture variations embedded throughout an entirety of the plurality of medical images.

19. The non-transitory computer readable storage media of claim 14, wherein learning simultaneously, via the vision transformer network, fine-grained features embedded in the plurality of medical images comprises:
providing the randomly distorted unique portion of each medical image that appears in each patch in the sequence of non-overlapping patches for each of the plurality of medical images to the vision transformer network; and
training the vision transformer network to recover the unique portion of each medical image that appears in each patch in the sequence of non-overlapping patches for each of the plurality of medical images.
